(19) United States
(12) Patent Application Publication
(10) Pub. No.: US 2014/0257173 A1
(43) Pub. Date: Sep. 11, 2014

(54) SYSTEM AND METHOD OF REDUCING ORAL BACTERIA

(71) Applicant: Vahe Ohanessian, Tujunga, CA (US)

(72) Inventor: Vahe Ohanessian, Tujunga, CA (US)

(21) Appl. No.: 14/193,255

(22) Filed: Feb. 28, 2014

Related U.S. Application Data
(60) Provisional application No. 61/866,187, filed on Aug. 15, 2013.
(60) Provisional application No. 61/774,031, filed on Mar. 7, 2013.

(51) Int. Cl. A61B 17/24; A61C 17/02; A61K 8/19; A61K 8/20; A61K 8/22; A61Q 11/00

(52) U.S. Cl. CPC A61B 17/24; A61K 8/22; A61Q 11/00; A61K 8/19; A61K 8/20; A61C 17/02
USPC 604/23; 424/53; 424/49; 424/51

(57) ABSTRACT

The present invention recognizes that there exists a need for the treatment diseases, disorders and conditions that cause or are related to bad breath in general. The present invention provides devices and methods to address this need. A first aspect of the present invention is an oral device for a subject. A second aspect of the present invention is a method of treating a subject for a disease, disorder, condition, or a combination thereof, of the oral cavity with a device of the present invention. A third aspect of the present invention is a method of treating a subject for a disease, disorder, condition, or a combination thereof, of the oral cavity.

The present application claims the benefit of priority to U.S. Provisional Patent Application Ser. No. 61/866,187, filed on Aug. 15, 2013, entitled “System and Method of Reducing Oral Bacteria,” and also U.S. Provisional Patent Application Ser. No. 61/774,031, filed on Mar. 7, 2013, entitled “System and Method of Reducing Oral Bacteria,” each of which are herein incorporated by reference in their entirety.

The present application also incorporates by reference in its entirety U.S. Provisional Patent Application Ser. No. 61/607,825, filed on Mar. 7, 2012, entitled “System and Method of Reducing Oral Bacteria,”

### TECHNICAL FIELD

The present invention relates to a system and method of treating bad breath, and more particularly to a system and method of reducing oral bacteria that are associated with bad breath and/or halitosis.

### BACKGROUND

Conventional systems and methods of treating bad breath, halitosis, and/or bacteria may try to eliminate bad breath through only mechanical scrapers, radiation, or mouthwash. Others may use mints, gum, or candy simply to mask the smell of bad breath. These conventional systems and methods may fail to significantly reduce the presence of oral bacteria associated with bad breath, and/or may only temporarily mask the odor of bad breath and/or halitosis.

Other conventional systems have been proposed. For example, US Patent Application No. 2009/0111069 to Wagner and US Patent Application No. 2006/0025791 to Ripich disclose using suction through a tongue scraper to collect debris and/or fluid. U.S. Pat. No. 6,689,078 to Rehkemper discusses using jets of fluid to clear out particles from between teeth. U.S. Pat. No. 7,544,204 to Krespi discloses irradiation of a patient's upper respiratory tract.

Each of these systems may also fail to significantly reduce the presence of oral bacteria associated with bad breath and/or halitosis, and conventional systems and methods may therefore be further improved.

### BRIEF DESCRIPTION OF THE DRAWINGS

FIG. 1 is a partially cutaway view of the system, according to an embodiment of the present invention;

FIG. 2A and FIG. 2B are cutaway views of the system, according to an embodiment of the present invention;

FIG. 3 is an illustration of an expanded width head of the system, according to an embodiment of the present invention;

FIG. 4 is an illustration of a restricted width head of the system, according to an embodiment of the present invention;

FIG. 5 is a flowchart illustrating operations of the method, according to an embodiment of the present invention; and

FIG. 6A and FIG. 6B are illustrations of systems that include a fluid dispensing material, according to embodiments of the present invention.

### SUMMARY

The present invention recognizes that there exists a need for the treatment diseases, disorders and conditions that cause or are related to bad breath in general. The present invention provides devices and methods to address this need.

A first aspect of the present invention is an oral device for a subject.

A second aspect of the present invention is a method of treating a subject for a disease, disorder, condition, or a combination thereof, of the oral cavity with a device of the present invention.

A third aspect of the present invention is a method of treating a subject for a disease, disorder, condition, or a combination thereof, of the oral cavity.

### DETAILED DESCRIPTION OF THE INVENTION

### Definitions

Unless defined otherwise, all technical and scientific terms used herein have the same meaning as commonly understood by one of ordinary skill in the art to which this invention belongs. Generally, the nomenclature used herein and the laboratory procedures in dentistry, microbiology, dental microbiology, oral microbiology; oral care and oral hygiene described herein are well known and commonly employed in the art. Conventional methods are used for these procedures, such as those provided in the art and various general references. Where a term is provided in the singular, the inventors also contemplate the plural of that term. Where a term is provided in the plural, the inventors also contemplate the singular of that term. The nomenclature used herein and the laboratory procedures described below are those well known and commonly employed in the art. As employed throughout the disclosure, the following terms, unless otherwise indicated, shall be understood to have the following meanings:

Other technical terms used herein have their ordinary meaning in the art that they are used, as exemplified by, but not limited to, a variety of technical dictionaries.

### Introduction

The present invention recognizes that there exists a need for the treatment diseases, disorders and conditions that cause or are related to bad breath in general. The present invention provides devices and methods to address this need.

As a non-limiting introduction to the breath of the present invention, the present invention includes several general and useful aspects, including:
1) an oral device for a subject;
2) a method of treating a subject for a disease, disorder, condition, or a combination thereof, of the oral cavity with a device of the present invention; and
3) a method of treating a subject for a disease, disorder, condition, or a combination thereof, of the oral cavity.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

The present invention generally pertains to the field of oral care. More specifically the invention pertains to an oral device of the present invention, a method of the present invention, or a combination thereof. More specifically, the current invention aims to treat bad breath.

### I. An Oral Device

The present invention includes an oral device for a subject.

A first aspect of the present invention is an oral device for a subject, including: a) at least one body; b) at least one scraping head; and c) one or more of 1) at least one gas; and 2) at least one reagent dispending material; wherein the at least one body and the at least one scraping head are operably engaged to apply physical contact of the at least one scraping head to the interior surfaces of a subject's oral cavity when in use.

A. Configuration of Body and Head Together and Separate

In another aspect of the present invention, the at least one body and the at least one scraping head are substantially hollow.

In the present invention, the at least one body and at least one scraping head can be, at least in part, either separately or in combination, substantially hollow such that at least one gas is able to flow through at least a portion of the device. Preferably, at least gas can enter the at least one body and exit the at least one scraping head. Preferably, the inside dimensions of the hollow interior of the oral device is such to permit the continuous flow of at least one gas through the at least one body and the at least one scraping head of the oral device of the present invention.

In the present invention, the at least one scraping head can be provided in a one or more configurations shaped in an anatomically designed way to engage the oral cavity of a subject. Preferably, each configuration of the at least one scraping head can be designed such that it specifically engages at least one of the surfaces of the oral cavity of a subject including, but not limited to, the surface of the tongue, the sides of the tongue, underneath the tongue, the interior surfaces of the cheeks, or the gums, or a combination thereof. The at least one scraping head can further include at least one ridged surface designed to disrupt at least one surface of the oral cavity of a subject when engaged therewith.

In the present invention, the at least one body and at least one scraping head can be unitary or made in pieces and assembled. The selection of the pieces can be any as desirable from a manufacture's point of view, such as but not limited to head, body, others, or a combination thereof. The pieces or unitary device can be made of the same or different materials. The unitary device or the pieces of the device can be substantially hollow, substantially solid, or a combination thereof. The pieces or unitary device can be made of any appropriate material, such as but not limited to plastics, polymers, metals, ceramics, glass, elastomers, thermoplastic elastomers, polycarbonates, high impact polystyrene, Acrylonitrate Butadiene Styrene, High Density Polyethylene, Glycol Infused Polyethylene Terephthalate, Recycled Polyethylene Terephthalate, Rigid Polyvinyl Chloride, Polymethyl Methacrylate, Co-extruded Plastics, silicone, or a combination thereof. The pieces or unitary device can be fabricated using appropriate methods depending on the type of materials. For example, for appropriate plastics and polymers, injection molding, milling, lathing, machining, or a combination thereof, can be used. Pieces can be assembled using methods appropriate for the materials at hand including, but not limited to, friction joint, snap fit, or sonic welding, thermal welding, melting, screw thread, cementing, adhesives, or a combination thereof.

In the present invention, the at least one body, at least one scraping head, or a combination thereof, can made of at least one of the following biocompatible materials, including but not limited to: (a) Polylactic acid (PLA), (b) poly-lactic-co-glycolic acid (PLGA), (c) polyglycolic acid (PGA), (d) polyethalene glycol (PEG), (e) Polycaprolactone (PCL), (f) Polydioxanone (PDO), (g) Poly (lactide-co-glycolide), (h) polyglyconate, (i) Polyhydroxybutyrate (PHB), (j) Polyhydroxyvalerate (PHV), (k) poly-pseudo amino acids, (l) Polyesteramides, (m) silk cocoons, (n) polyanhydrides, (o) polyorthoesters, (p) polyphosphazones, (q) Therpmoplastic elastomers, (r) including but not limited to polyether block amide (PEBA), (s) Fluoropolymers, (t) silicone, (u) latex, (v) EDTA, (w) animal epithelia, (x) spider or cocoon silk, (y) nano-materials, (z) Poly(ethylene-covinyl acetate), (aa) Hydrophilic polyether urethane (HPEU), (bb) Polyether urethane (PEU), (cc) osmotic agents (e.g. NaCl or Sodium Acetate), (dd) Polyhydroalkanoates (PHAs), such as (ee) Polyhdroxyhexanoate (PHH), (ff) Polybutylene succinate (PBS), (gg) Polycaprolactone (PCL), (hh) cellulose esters, (ii) polyvinyl alcohol, or a combination thereof.

In the present invention, the at least one body, at least one scraping head, or a combination thereof, can made of at least one of the following materials, including but not limited to: (a) Low Density Polyethylene, (b) High Density Polyethylene, (c) Celluloze Acetate, (d) Chlorinated Polyethylene, (e) Ethylene-vinyl Acetate, (f) Ethylene-Ethylacrylate, (g) Ethylene-Methylacrylic Acid, (h) Polypropylene battery, (i) Polyamide 6, (j) Polyamide 66, (k) polyoxymethylene, (l) General Purpose Polystyrene, (m) High Impact Polystyrene, (n) Expendable Polystyrene, (o) Styrene-Acrylo-Nitrile, (p) Acrylonitrile-butadiene-styrene, (q) Methylmethacrylate-butadiene-styrene, (r) Styrene-butadiene, (s) Polymethyl Methacrylate, (t) Polycarbonate, (u) Polyvinyl Chloride, (v) Polyvinyl Formal, (w) Polyvinyl Butyral, (x) Polyethylene Terephthalate, (y) Polybutylene Terephthalate, (z) Epoxy Resin, (aa) Fluoreod Plastics, (bb) Polyphenylene Sul de, (cc) Polyphenylene Oxide, (dd) Butadiene Rubber, (ee) Styrene-Butadiene-styrene, or a combination thereof.

In a further aspect of the present invention, the at least one scraping head is detachable from the at least one body of said oral device.

In the present invention, the at least one scraping head can be reversibly detachable, irreversibly detachable, or permanently engaged, from the at least one body of the oral device. Preferably, the at least one scraping head can be reversibly detachable, or irreversibly detachable from the at least one body of the oral device by at least one mechanism including, but not limited to: a friction joint, a “snap fit,” sonic welding, compression fitting, screw thread, cementing, adhesives, or a combination thereof, or a combination thereof. The at least one scraping head can be designed with key sealing, washer sealing, or a combination thereof, at the articulating end that fits into the socket of the at least one body of the oral device, such that it permits the proper alignment of the at least one scraping head onto the at least one body of the oral device. The key sealing, washer sealing, or a combination thereof, can also permit fastening of the at least one scraping head into the at least one body of the oral device of the present invention. The key sealing, washer sealing, or a combination thereof, can promote a more secure attachment, and minimize the amount of gas loss, between the joint of the at least one body and at least one scraping head.

In an additional aspect of the present invention, the at least one scraping head includes at least one aperture.

In the present invention, the at least one aperture can be a part of the at least one scraping head, separate from the at least one scraping head, on at least one vertical plane, can be staggered horizontally, vertically, or a combination thereof. More specifically, the at least one aperture can be present on the posterior surface of the at least one scraping head, the anterior surface of the at least one scraping head, the lateral surfaces of the at least one scraping head, or a combination thereof. Preferably, the at least one aperture can be one size or a range of sizes; can be at least one shape, or a combination of shapes. The at least one aperture can be in-between at least one protrusion on the at least one scraping head, can be incorporated into the at least one scraping head, can be separate from the at least one scraping head, or a combination thereof. The at least one aperture permits flow of at least one gas through the oral device of the present invention. Preferably, the at least one aperture permits the flow of at least one gas to the at least one oral surface of a subject, when engaged therewith.

B. At Lease One Gas

In another aspect of the present invention, the oral device allows the flow of the at least one gas into the at least one body and exits from the at least one scraping head.

In the present invention, the at least one gas can exit from the at least one aperture on the at least one scraping head, and preferably enters into the body. However, other configurations such as the at least one gas entering into and exiting from the head.

In the present invention, the oral device can be engaged with at least one external gas source that allows the flow of at least one gas into the device and then exits the device, such but not limited to entering the at least one body and exiting from the at least one scraping head. Preferably, the oral device can engage with at least one external gas source through at least one linkage including, but not limited to: at least one tube, polymer tube, at least one metallic tube, at least one “Y-connector,” at least one valve joint, at least one valve, or a combination thereof. Preferably, the at least one linkage can engage with the at least one external gas sources and the at least one body, at least one scraping head, or a combination thereof, of the oral device, such that gas flows from the at least one external gas source into the oral device. The at least one gas can flow at a rate preferable of between about 0.1 psi and about 50,000 psi, more preferably between about 1 psi and about 1,000 psi, and more preferably between about 10 psi and about 100 psi, though other ranges and values are appropriate and applicable to the present invention. The at least one gas includes, but is not limited to: oxygen, hyperbaric oxygen, nitrogen, nitrogen dioxide, hydrogen, helium, carbon dioxide, compressed air, nitrous oxide, or a combination thereof.

In the present invention, the at least one oral device can have at least one method of internally generating a flow of at least one gas through the oral device. Preferably, the at least one method of internally generating a flow of at least one gas through the oral device includes, but is not limited to: a propeller, a suction force, compressed gas cartridge, servile motor driven, electrically driven, solar driven, heat gradient driven, gas gradient driven, or a combination thereof. Preferably, the flow of at least one gas can enter into the at least one body and exits from the at least one scraping head of the oral device. The at least one method of internally generating a flow of at least one gas through the oral device can be activated when engaged by a subject, engaged by at least one surface of the oral cavity of a subject, or a combination thereof. The oral device can include at least one aperture that allows for an influx of at least one gas, including ambient air, into the at least one body and exiting through the at least one scraping head of the oral device

In a further aspect of the present invention, the at least one gas includes oxygen.

In the present invention, the at least one gas also includes hyperbaric oxygen, nitrogen, nitrogen dioxide, hydrogen, helium, carbon dioxide, compressed air, nitrous oxide, or a combination thereof. Each of the gases, alone, or in any combination, is preferably present at a partial pressure, amount, or a combination thereof, such that it is determined as effective and safe for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof.

In the present invention, the at least one gas can be projected into the oral cavity of a subject individually, or in combination with other gases. More specifically, the at least one gas can be projected individually, or in combination with other gases to achieve a desired result, depending on the intended use by a subject, practitioner, or other user. Preferably, one use can be projecting at least one gas, including oxygen, to at least one surface of the oral cavity of a subject, for a predetermined or sufficient amount of time for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof. Preferably, the at least one gas can be present in an effective and safe amount, and delivered for an appropriate or predetermined amount of time sufficient to affect at least one oral bacterium.

C. Scraping Head with External Reagent Dispensing Material

In another aspect of the present invention, the at least one reagent dispensing material faces generally outward from the oral device, the at least one body, the at least one scraping head, or a combination thereof.

In the present invention, the at least one reagent dispensing material preferably faces generally outward from at least one surface of the at least one scraping head of the oral device. More specifically, the at least one reagent dispensing materials is preferably positioned such that it engages at least one surface of the oral cavity of a subject, when engaged therewith. More specifically, the at least one reagent dispensing material is preferably positioned at or towards the tip of the contact surface, distal from the contact surface, or a combination thereof, of the at least one scraping head, at the anterior surface of the at least one scraping head, at the posterior surface of the at least one scraping head, or a combination thereof.

In a further aspect of the present invention, the at least one reagent dispensing material includes: at least one sponge, at least one hydrogel, at least one hydrogel with at least one dissolved gas, at least one hydrophilic rubber, at least one fabric, or a combination thereof.

In the present invention, the at least one reagent dispensing material can include at least one sponge or sponge-like material. Preferably, at least one reagent can be embedded within the at least one sponge, on the anterior surface of the at least one sponge, the posterior surface of the at least one sponge, or a combination thereof. Preferably, at least one reagent is embedded within or coated around the sponge or sponge-like material. More specifically, the at least one reagent is preferably embedded within or coated around the at least one sponge or sponge-like material, or a combination thereof, such that it allows dispensing of the at least one reagent onto at least one surface of the oral cavity of a subject, when engaged therewith.

In the present invention, the at least one reagent dispensing material can include at least one hydrogel. Preferably, the at least one hydrogel can include a therapeutically safe and effective amount of at least one biodegradable hydrogel including, but not limited to: at least one polymer, at least one hydrophilic polymer, at least one essential oil, or a combination thereof. Preferably, the at least one polymer can include, but is not limited to: polysaccharides, beta-linked acetylated mannan, Xylitol, glyceryl acrylate, glyceryl polyacrylate, chitosan, or a combination thereof; the at least one hydrophilic polymer can include, but is not limited to: Poly(N-isopropylacrylamide) (PNIPAM) and Polyacrylamide (PAM), Poly(2-oxazoline) and Polyethylenimine (PEI), Poly(acrylic acid), Polymethacrylate and Other Acrylic Polymers, Poly(ethylene glycol) and Poly(ethylene oxide), Poly(vinyl alcohol) (PVA) and Copolymers, Poly(vinylpyrrolidinone) (PVP) and Copolymers, Polyelectrolytes, Cucurbit[n]uril Hydrate, or a combination thereof the at least one essential oil can include, but is not limited to: oil of cinnamon, oil of thyme, oil of clove, or a combination thereof. Preferably, the at least one hydrogel can include at least one reagent embedded within or coated onto the surface of the at least one hydrogel, or a combination thereof. Preferably, the at least one reagent is embedded within or coated around the at least one hydrogel, or a combination thereof, such that it allows dispensing of the at least one reagent onto at least one surface of the oral cavity of a subject when used by a subject. The at least one hydrogel can include at least one dissolved gas, such that the at least one gas would be released onto at least one surface of the oral cavity of a subject, when engaged therewith.

In the present invention, the at least one reagent dispensing material can include at least one rubber, at least one hydrophobic rubber or hydrophilic, at least one hydrophilic rubber, or a combination thereof. Preferably, the at least one hydrophilic rubber can include, but is not limited to: Acrylonitrile Butadiene (NBR) Elastomers, nitrile rubber, carboxylated nitrile rubber, nitrile/PVC polyblends, plasticized nitrile rubber, Acrylonitrile-butadiene lactices, ethelyeneoxide based polyethers, polyacrylic rubber, other plasticizers, or a combination thereof. Preferably, at least one reagent can be embedded within or coated onto the at least one hydrophilic rubber, or a combination thereof, such that it allows dispensing of the at least one reagent onto at least one surface of the oral cavity of a subject, when engaged therewith.

In the present invention, the at least one reagent dispensing material can include at least one fabric. Preferably, at least one reagent can be embedded within or coated onto the at least one fabric, or a combination thereof. More specifically, the at least one reagent is preferably embedded within or coated around the at least one fabric, or a combination thereof, such that it allows dispensing of the at least one reagent onto at least one surface of the oral cavity of a subject, when engaged therewith.

C. Scraping Head with Internal Liquid Phase Dispensing Material

In another aspect of the present invention, the internal space of the oral device, the at least one body, the at least one scraping head, or a combination thereof, includes at least one liquid phase.

In the present invention, the internal space of the oral device, the at least one body, the at least one scraping head, or a combination thereof, preferably includes at least one reservoir containing at least one liquid phase reagent located proximate to the at least one reagent dispensing material. More specifically, the at least one reservoir containing at least one liquid phase reagent can be engaged by a user allowing the at least one liquid phase reagent to be dispensed into, onto, or both, the at least one reagent dispensing material, saturating the at least one reagent dispensing material. More specifically, the saturated at least one reagent dispensing material can dispense the at least one liquid phase reagent onto at least one surface of the oral cavity of a subject when engaged within the oral cavity of a subject.

In a further aspect of the present invention, the at least one liquid phase includes: at least one antiseptic, at least one anti-microbial, at least one anti-fungal, at least one halogen-releasing antiseptic, at least one freshening agent, at least one bleach, at least one enzyme, at least one iodine, at least one numbing agent, or a combination thereof.

In the present invention, the at least one liquid phase preferably includes at least one antiseptic or at least one halogen-releasing antiseptic, or a combination thereof. The at least one antiseptic or at least one halogen-releasing antiseptic, or a combination thereof, can include a therapeutically effective amount of orally safe antiseptics preferably for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof, including, but not limited to: (a) Cetyl Pyridinium Chloride Ph Eur, (b) alcohol, (c) hydrogen peroxide, (d) chlorine, (e) chlorine dioxide, (f) sodium hypochlorite or other chlorine based compounds, (g) chlorhexidine, (h) chlorhexidine gluconate or di-gluconate, (i) chlorhexidine acetate, (j) iodine, (k) iodophors or other iodine-based compounds, (l) hexetidine alcohol, (m) cetrimide, (n) benzethonium chloride or other Quanternary ammonium chlorides (QAC's), (o) Dequalinium chloride, (p) triclosan, (q) zinc sulphate, (r) coconut water, (s) coconut oil, (t) tea tree oil, (u) other organic antiseptics, (v) sodium lauryl sulphate (SLS), or a combination thereof.

In the present invention, the at least one liquid phase preferably includes at the at least one antimicrobial. The at least one antimicrobial includes a therapeutically effective amount of orally safe therapeutic antibiotics preferably for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof, including, but not limited to: (a) amoxicillin, (b) ampicillin, (c) clindamycin, (d) erythromycin, (e) metronidazole, (f) penicillin, (g) gentamicin, (h) ampicillin-sulbactam, (i) cefoxitin, (j) cefotetan, (k) cefazolin, (l) ciprofloxacin, (m) rifampin, (n) vancomycin, (o) azithromycin, (p) ampicillin-clavulanic acid (q) cephalosporins, (r) levofloxacin, (s) quinolones, (t) chloramphenicol, (u) nitrofurantoin, (v) cefuroxime, (w) cefaclor, (x) cefadroxil, (y) cefamandole, (z) cefpodoxime, (aa) cefprozil, (bb) ceftazidime, (cc) ceftibuten, (dd) ceftizoxime, (ee) ceftriaxone, (ff) cephalexin, (gg) cephapirin, (hh) cephradine, (ii) cefdinir, (jj) cefditoren, (kk) cefepime, (ll) cefixime, (mm) sulfamethoxazole/trimethoprim, or a combination thereof.

In the present invention, the at least one liquid phase preferably includes at the at least one antifungal including a therapeutically effective amount of antifungal agents preferably for the purpose of inhibiting growth of, reducing the number of, or killing of, at least one type of at leas one type of fungus, at least one type of yeast, at least one type of microorganism, or a combination thereof, including, but not limited to: (a) imidazole; (b) triazole; (c) thiazole, or similar agents, or a combination thereof.

In the present invention, the at least one liquid phase preferably includes at the at least one freshening agent. The at least one freshening agent preferably includes a therapeutically effective amount of orally safe freshening agents able to preferably have a freshening affect including, but not limited to: (a) cinnamon supra, (b) green tea base, (c) parsley herb, (d) peppermint American Far W., (e) spearmint American Far W., (f) ‘Boudicca’, (g) ‘Theseus’, (h) Hearakles', or a combination thereof.

In the present invention, the at least one liquid phase preferably includes at the at least one bleach. The at least one bleach preferably includes a therapeutically effective amount of orally safe bleach, preferably at a concentration between about 0.1% (w/w) and about 10% (w/w), more preferably between about 3% (w/w) and about 6% (w/w), though other values are appropriate and applicable to the present invention, preferably for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof, including, but not limited to: (a) hydrogen peroxide, (b) carbamide peroxide, (c) 6-phthalimido peroxy hexanoic acid (PAP), or a combination thereof.

In the present invention, the at least one liquid phase includes at the at least one oral numbing agent. The at least one oral numbing agent comprises a therapeutically effective amount of the numbing agent in order to achieve a noticeable numbing affect including, but not limited to: (a) benzocaine, (b) lidocaine, (c) butamben, (d) dibucaine, (e) oxybuprocaine, (f) pramoxine, (g) proparacaine, (h) proxymetacaine, (i) tetracaine (amethocaine), or a combination thereof.

D. Certain Preferred Aspects of the Present Invention

In another aspect of the present invention, the oral device includes at least one gas, at least one solution, at least one chemical, or a combination thereof.

In the present invention, the at least one gas includes: oxygen, hyperbaric oxygen, nitrogen, nitrogen dioxide, hydrogen, helium, carbon dioxide, compressed air, nitrous oxide, or a combination thereof. Each of the gases, alone, or in any combination, is present at a partial pressure, amount, or a combination thereof, such that it is recognized as effective and safe.

Preferably, the at least one gas can be present in an effective and safe amount, preferably at a lower range between about 0.1 mmHg and about 30 mmHg and a higher range between about 30 mmHg and about 150 mmHg, more preferably between about 15 mmHg and about 30 mmHg, though other values and ranges are applicable to the present invention. Preferably, the at least one gas can be delivered for a predetermined amount of time, preferably ranging from a between about 0.1 seconds and about 180 minutes, more preferably ranging between about 1 minute and about 10 minutes, though other values and rangers are applicable to the present invention.

Preferably the at least one gas can be delivered at the effective and safe amount and predetermined amount of time, preferably for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof.

In the present invention, the at least one solution or at least one chemical preferably includes: at least one antiseptic, at least one anti-microbial, at least one anti-fungal, at least one halogen-releasing antiseptic, at least one freshening agent, at least one bleach, at least one chloride, at least one enzyme, at least one iodine, at least one numbing agent, or a combination thereof. Preferably, the at least one chemical being relatively non-toxic to the subject at the locus, dose, and route of administration used.

More preferably the at least one antiseptic, at least one antimicrobial, at least one antifungal, or at least one halogen-releasing antiseptic includes: chlorhexidine, chlorhexidine gluconate, chlorhexidine digluconate, chlorine dioxide, sodium hypochlorite or other chlorine based compounds, iodophors or other iodine-based compounds, hydrogen peroxide, hexetidine, alcohol, or a combination thereof.

In a further aspect of the present invention, the oral device drives the projection of the at least one gas, at least one solution, at least one chemical, or a combination thereof, away from said oral device towards the interior of the subject's oral cavity upon contact therewith.

In the present invention, the projection of at least one gas away from said oral device towards the interior of the subject's oral cavity upon contact therewith, can include: engaging the oral device with at least one external gas source, providing at least one reservoir with at least one prefilled gas, or a combination thereof. More specifically, the at least one reservoir with at least one prefilled gas can be released when engaged with the oral device. More preferably, the at least one reservoir with at least one prefilled gas can be released when a subject engages the oral device.

In the present invention, the projection or projections of at least one gas away from said oral device towards the interior of the subject's oral cavity upon contact therewith, can include at least one internal, external, or combination thereof, mechanism of generating or producing at least one gas flow. Preferably, the at least one internal means of generating at least one gas flow preferably includes at least one propeller. Preferably, the at least one motor can be activated when a subject engages the oral device. Preferably, the at least one motor can be designed such that it generates flow of at least one gas into the at least one body and exit from the at least one scraping head of the oral device when engaged by a subject.

In the present invention, the projection of at least one solution, at least one chemical, or a combination thereof, away from said oral device towards the interior of the subject's oral cavity upon contact therewith, can include but is not limited to: engaging at least one reagent dispensing material containing at least one solution with at least one surface of the oral cavity of a subject. More specifically, the at least one reagent dispensing material containing at least one reagent can dispense a controlled amount of at least one reagent to at least one surface of the oral cavity of a subject, when engaged therewith.

In the present invention, the projection of at least one solution, at least one chemical, or a combination thereof, away from said oral device towards the interior of the subject's oral cavity upon contact therewith, can include: a subject engaging the at least one liquid phase reservoir; the at least one liquid phase reservoir subsequently engaging with the at least one reagent dispensing material; and the at least one liquid phase and at least one reagent dispensing material dispensing at least one reagent when engaged with the oral cavity of a subject. More specifically, the at least one liquid phase and the at least one reagent dispensing material containing at least one reagent can dispense a controlled amount of at least one reagent to at least one surface of the oral cavity of a subject, when engaged therewith.

### II. Method of Using a Device of the Present Invention

The present invention also includes a method of treating a subject for a disease, disorder, condition, or a combination thereof, of the oral cavity.

A first aspect of the present invention is a method of treating a subject for a disease, disorder, condition, or a combination thereof, of the oral cavity, including; a) providing a subject in need of treatment of a disease, disorder, condition, or a combination thereof, of the oral cavity; b) providing at least one oral device of the present invention; and c) engaging the subject with the at least one oral device; wherein the subject is treated for said disease, disorder, condition, or a combination thereof, of the oral cavity.

A. Subject

In another aspect of the present invention, the subject is a mammal, a primate, a non-human primate, or a human.

In the present invention, the subject can include: a research-testing animal, a non-research testing animal, a mammal primate, non-human primate, a clinical testing mammal, a veterinary animal, or a human Preferably, the subject can benefit from using the oral device of the present invention, receiving the treatment method of the present invention, or a combination thereof.

B. Disease, Disorder, Condition, or a Combination Thereof

In another aspect of the present invention, the disease, disorder, condition, or a combination thereof, includes bad breath, halitosis, chronic halitosis, calculus, periodontitis, gum disease, fungal infections, dry mouth, or a combination thereof.

In the present invention, the oral device, the treatment method, or a combination thereof, of the present invention can be utilized to treat at least one disease, disorder, condition, or a combination thereof. More specifically, the oral device, the treatment method, or a combination thereof, of the current invention can be utilized to treat bad breath, halitosis, or chronic halitosis, ranging from everyday bad breath, morning breath, halitosis, chronic halitosis, or a combination thereof. Engaging the oral device of the present invention in the manner described herein, or any such manner, which would be readily apparent to one of ordinary skill in the art, can be used to treat bad breath, halitosis, chronic halitosis, or a combination thereof.

In the present invention, the oral device, the treatment method, or a combination thereof, of the present invention can be utilized to treat at least one disease, disorder, condition, or a combination thereof. More specifically, the oral device, the treatment method, or a combination thereof, of the current invention can be utilized to treat calculus, periodontitis, gum disease, or a combination thereof, ranging from calculus removal, treatment of periodontitis, treatment of gum disease, or a combination thereof. Engaging the oral device of the present invention in the manner described herein, or any such manner, which would be readily apparent to one of ordinary skill in the art, can be used to treat calculus, periodontitis, gum disease, or a combination thereof. More specifically, engaging the oral device of the present invention with the gingival tissues of a subject can be used to treat calculus, periodontitis, gum disease, or a combination thereof.

In the present invention, the oral device, the treatment method, or a combination thereof, of the present invention can be utilized to treat at least one disease, disorder, condition, or a combination thereof. More specifically, the oral device, the treatment method, or a combination thereof, of the current invention can be utilized to treat at least one fungal infection including but not limited to, oral thrush. Engaging the oral device of the present invention in the manner described herein, or any such manner, which would be readily apparent to one of ordinary skill in the art, can be used to treat bad breath, halitosis, chronic halitosis, or a combination thereof.

In a further aspect of the present invention, the disease, disorder, condition, or a combination thereof, includes bad breath.

In the present invention, the oral device, the treatment method, or a combination thereof, of the current invention can be utilized to treat at least one disease, disorder, condition, or a combination thereof, including bad breath. More specifically, utilizing the oral device of the present invention, the treatment method of the present invention, or a combination thereof, to treat at least one disease, disorder, condition, or a combination thereof, including bad breath. More specifically, engaging at least one surface of the oral cavity of a subject with the oral device of the present invention can help treat bad breath. More specifically, engaging at least one surface of the oral cavity of a subject with the oral device of the present invention for a predetermined period of time can help treat bad breath. More specifically, engaging at least one surface of the oral cavity of a subject with the oral device of the present invention at least once per day, for a sufficient or predetermined period of time can help treat bad breath.

C. Engaging Subject With Device

In another aspect of the present invention, the subject's oral cavity is engaged with the at least one oral device.

In the present invention, the at least one oral device is used by a subject to engage the subject's own at least one surface of the oral cavity, or at least one surface of the oral cavity of a different subject. Preferably a subject or other user guides the at least one oral device to at least one surface of the subjects own oral cavity, or at least one surface of the oral cavity of a different subject, to engage it therewith.

In another aspect of the present invention, the subject's oral cavity is contacted with the at least one oral device.

In the present invention, the at least one oral device can be used by a subject or other user to contact at least one surface of the subject's oral cavity. Preferably a subject or other user applies physical pressure with at least one oral device to at least one surface of the subject's oral cavity, to promote physical disruption of the at least on surface of the oral cavity, when contacted therewith. Preferably, the contact can be a scraping motion in any direction including, but not limited to: a relative or substantial forward motion, a relative or substantial backward motion, a relative or substantial lateral motion, relative or substantial circular motion, or a combination thereof.

In another aspect of the present invention, the at least one oral device administers at least one gas, at least one solution, at least one chemical, or a combination thereof to the oral cavity of said subject.

In the present invention, the at least one oral device preferably dispenses at least one reagent from the at least one liquid phase reservoir or at least one reagent dispensing material, or a combination thereof, when engaged and contacted with at least one surface of the oral cavity of a subject. The at least one oral device can preferably also simultaneously deliver at least one gas, or not deliver at least one gas, while engaged and contacted with at least one surface of the oral cavity of a subject. Preferably, the at least one oral device can be used by a subject or other user to administer at least one gas, at least one solution, at least one chemical, or a combination thereof, to at least one surface of the subjects oral cavity.

### III. Method of Treating a Subject

The present invention includes a method of treating a subject for a disease, disorder, condition, or a combination thereof, of the oral cavity.

A first aspect of the present invention is a method of treating a subject for a disease, disorder, condition, or a combination thereof, of the oral cavity, including; a) providing a subject in need of treatment of a disease, disorder, condition, or a combination thereof, of the oral cavity; b) providing at least one gas efficacious for the treatment of the disease, disorder, condition, or a combination thereof, of the oral cavity; and c) providing at least one chemical efficacious for the treatment of the disease, disorder, condition, or a combination thereof, of the oral cavity; wherein the subject is treated for the disease, disorder, condition, or a combination thereof, of the oral cavity.

In another aspect of the present invention, the at least one gas includes oxygen, hyperbaric oxygen, nitrogen, nitrogen dioxide, hydrogen, helium, carbon dioxide, compressed air, nitrous oxide, or a combination thereof.

In the present invention, the at least one gas also includes hyperbaric oxygen, nitrogen, nitrogen dioxide, hydrogen, helium, carbon dioxide, compressed air, nitrous oxide, or a combination thereof. Each of the gases, alone, or in any combination, is preferably present at a partial pressure, amount, or a combination thereof, such that it is determined as effective and safe for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof.

Preferably, the at least one gas can be present in an effective and safe amount, preferably at a lower range between about 0.1 mmHg and about 30 mmHg and a higher range between about 30 mmHg to about 150 mmHg, more preferably between about 15 mmHg and about 30 mmHg, though other values and ranges are applicable to the present invention. Preferably, the at least one gas can be delivered for a predetermined amount of time, preferably ranging from between about 0.1 seconds to about 180 minutes, more preferably ranging between about 1 minute and about 10 minutes, though other values and rangers are applicable to the present invention.

Preferably the at least one gas can be delivered at the effective and safe amount and predetermined amount of time, preferably for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof, in the oral cavity of a subject, when engaged therewith.

In the present invention, the at least one gas can be projected into the oral cavity of a subject individually, or in combination with other gases. More specifically, the at least one gas can be projected individually, or in combination with other gases to achieve a desired result, depending on the intended use by a subject, practitioner, or other user. One use can be projecting at least one gas, including oxygen, to at least one surface of the oral cavity of a subject, for a predetermined or sufficient amount of time for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof, in the oral cavity of a subject, when engaged therewith. Preferably, the at least one gas can be present in an effective and safe amount, and delivered for an appropriate or predetermined amount of time sufficient to for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof, in the oral cavity of a subject, when engaged therewith.

In a further aspect of the present invention, the at least one chemical includes at least one antiseptic, at least one antifungal, at least one antibiotic, at least one bleach, at least one iodine, at least one alcohol, at least one freshening agent, at least one hydrogen peroxide, at least one moisturizing agent, at least one salivating agent, at least one wetting agent, at least one numbing agent, or a combination thereof.

In the present invention, the at least one liquid phase preferably includes at least one antiseptic or at least one halogen-releasing antiseptic, or a combination thereof. The at least one antiseptic or at least one halogen-releasing antiseptic, or a combination thereof, can preferably include a therapeutically effective amount of orally safe antiseptics preferably for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof, in the oral cavity of a subject, when engaged therewith, including, but not limited to: (a) Cetyl Pyridinium Chloride Ph Eur, (b) alcohol, (c) hydrogen peroxide, (d) chlorine, (e) chlorine dioxide, (f) sodium hypochlorite or other chlorine based compounds, (g) chlorhexidine, (h) chlorhexidine gluconate or di-gluconate, (i) chlorhexidine acetate, (j) iodine, (k) iodophors or other iodine-based compounds, (l) hexetidine alcohol, (m) cetrimide, (n) benzethonium chloride or other Quanternary ammonium chlorides (QAC's), (o) Dequalinium chloride, (p) triclosan, (q) zinc sulphate, (r) coconut water, (s) coconut oil, (t) tea tree oil, (u) other organic antiseptics, (v) sodium lauryl sulphate (SLS), or a combination thereof.

In the present invention, the at least one liquid phase includes at the at least one antimicrobial. The at least one antimicrobial includes a therapeutically effective amount of orally safe therapeutic antibiotics preferably for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof, in the oral cavity of a subject, when engaged therewith, including, but not limited to: (a) amoxicillin, (b) ampicillin, (c) clindamycin, (d) erythromycin, (e) metronidazole, (f) penicillin, (g) gentamicin, (h) ampicillin-sulbactam, (i) cefoxitin, (j) cefotetan, (k) cefazolin, (l) ciprofloxacin, (m) rifampin, (n) vancomycin, (o) azithromycin, (p) ampicillin-clavulanic acid (q) cephalosporins, (r) levofloxacin, (s) quinolones, (t) chloramphenicol, (u) nitrofurantoin, (v) cefuroxime, (w) cefaclor, (x) cefadroxil, (y) cefamandole, (z) cefpodoxime, (aa) cefprozil, (bb) ceftazidime, (cc) ceftibuten, (dd) ceftizoxime, (ee) ceftriaxone, (ff) cephalexin, (gg) cephapirin, (hh) cephradine, (ii) cefdinir, (jj) cefditoren, (kk) cefepime, (ll) cefixime, (mm) sulfamethoxazole/trimethoprim, or a combination thereof.

In the present invention, the at least one liquid phase includes at the at least one antifungal including a therapeutically effective amount of antifungal agents preferably for the purpose of inhibiting growth of, reducing the number of, or killing of, at leas one type of fungus, at least one type of yeast, at least one type of microorganism, or a combination thereof, in the oral cavity of a subject, when engaged therewith, including, but not limited to: (a) imidazole; (b) triazole; (c) thiazole, or similar agents, or a combination thereof.

In the present invention, the at least one liquid phase includes at the at least one freshening agent. The at least one freshening agent includes a therapeutically effective amount of orally safe freshening agents able to have a freshening affect including, but not limited to: (a) cinnamon supra, (b) green tea base, (c) parsley herb, (d) peppermint American Far W., (e) spearmint American Far W., (f) ‘Boudicca’, (g) ‘Theseus’, (h) ‘Hearakles’, or a combination thereof.

In the present invention, the at least one liquid phase preferably includes at the at least one bleach. The at least one bleach preferably includes a therapeutically effective amount of orally safe bleach, preferably at a concentration between about 0.1% (w/w) and about 10% (w/w), more preferably between about 3% (w/w) and about 6% (w/w), though other values are appropriate and applicable to the present invention, preferably for the purpose of inhibiting growth of, reducing the number of, or killing of, at least one type of gram-positive bacterium, at least one type of gram-negative bacterium, at least one type of anaerobic bacterium, at least one type of aerobic bacterium, at leas one type of fungus, at least one type of yeast, at least one type of microorganism, at least one type of bacterial biofilm, or a combination thereof, including but not limited to: (a) hydrogen peroxide, (b) carbamide peroxide, (c) 6-phthalimido peroxy hexanoic acid (PAP), or a combination thereof.

In the present invention, the at least one liquid phase includes at the at least one oral numbing agent. The at least one oral numbing agent comprises a therapeutically effective amount of the numbing agent in order to achieve a noticeable numbing affect including, but not limited to: (a) benzocaine, (b) lidocaine, (c) butamben, (d) dibucaine, (e) oxybuprocaine, (f) pramoxine, (g) proparacaine, (h) proxymetacaine, (i) tetracaine (amethocaine), or a combination thereof.

In an additional aspect of the present invention, the method utilizes at least in part a device, utilizes at least in part no device, no device, or a combination thereof.

In the present invention, the method of treatment preferably utilizes at least in part the at least one oral device described herein to provide the treatment of the present invention. The present method of treatment can utilize at least in part the device of the present invention, at least in part a device not of the present invention, or a combination thereof, to provide the treatment method of the present invention.

### EXAMPLES

The system and method treats oral bacteria that often are a source of bad breath and/or halitosis. The oral bacteria may be located in multiple areas of the mouth, such as the tongue, the cheeks, the gums, and the roof of a person's mouth. Large concentrations of the oral bacteria may be found in the papillae on a person's tongue.

The system and method provides an improved treatment of bad breath and/or halitosis by more effectively exposing the oral bacteria to oxygen through one or more of mechanical action involving scrapers and/or brushes, simultaneous positive gas flow that includes oxygen, and/or a gas flowing treatment. The system may include the use of specialized devices to assist in the exposure of oral bacteria to oxygen, air, chemicals, and/or solutions that are described in greater detail below in connection with the figures.

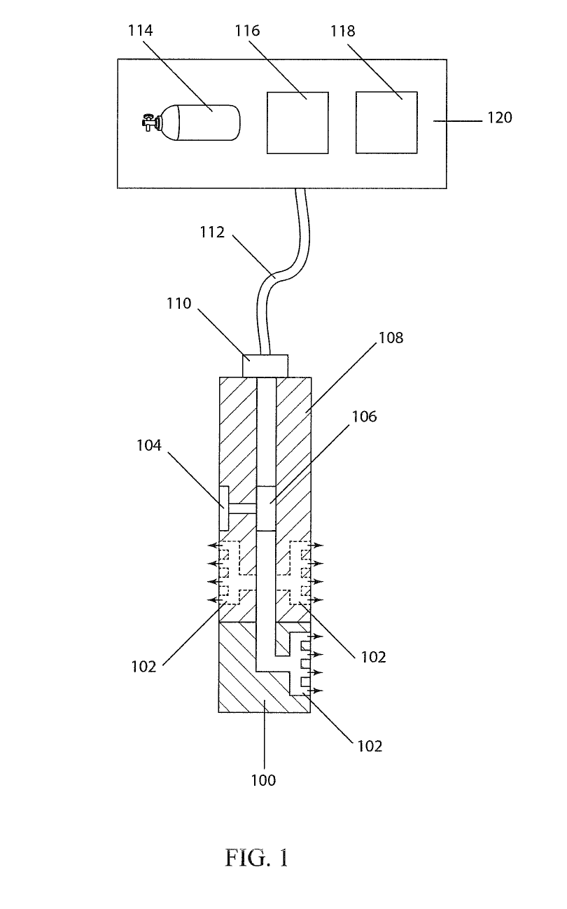

FIG. 1 illustrates the system according to an embodiment. The system includes a body (108) suitable for gripping by a user's hand. The body may extend parallel to a first axis, e.g., the X-axis. The system may also include a head (100) that is removable or integrally formed with the body. The head may include a channel for conducting gas, and at least one outlet (102) for emitting gas into a person's mouth to treat oral bacteria. The gas includes oxygen, and in a most preferred embodiment consists of or consists essentially of pure oxygen. The gas may simply be air taken from the environment or may be air mixed with additional oxygen so that the oxygen concentration is substantially above (twenty percent or more) that of air.

In a most preferred embodiment, the head (100) may include at least one surface, and a plurality of outlets disposed on the surface. The outlet(s) may be disposed such that gas directed out of the outlets is projected with a directional component that is perpendicular to the X-axis. In other embodiments, the gas may be directed parallel to the X-axis. Other angles of emitting the air from the head may be used as well.

The outlet(s) may be disposed around and/or on surface features of the head, such as protrusions, ridges, dimples, brushes, bristles, and/or indentations (not shown). The surface features may be soft or hard, rounded or pointed. The surface features may be formed for rubbing against the tissue surfaces of the interior of a person's mouth, such as the upper, side, and lower surfaces of the tongue, the inner walls of the cheeks, the roof of a person's mouth, and/or a person's gums.

The body (108) may include a surface formed with additional outlets. The additional outlets may be disposed on and/or around surface features of the body, and may be oriented to project air out from the body with a vector component that is parallel to the X-axis and/or the Y-axis. The body may include a valve (106) for controlling gas flow into the body before it is projected out and thus into a user's mouth. The body may further include a control interface (104), such as a push button, a switch, or a dial, that controls whether the valve opens or closes. The control interface and the valve may be mechanically or electrically coupled, and the opening and closing of the valve may be driven by the control interface or by a motor. The body may be formed with a channel for directing gas out through the outlets of the head and/or body and into a person's mouth. In some embodiments, the body may lack the control interface and valve, and may simply be used to direct gas from the source to a person's mouth.

The body may be coupled to a source of pressurized gas (114) through a connector (110) and a hose or pipe (112). The gas may be at a higher pressure than the ambient atmospheric pressure. The source (120) may include one or more of compressed gas (114), a compressor (116), and a pipeline (118), such as for a building or vehicle. The pipeline may in turn be connected to a separate storage location for compressed gas and/or a gas compressor.

The head may be used to deliver gas to the interior of a person's mouth while scraping, or brushing, massaging, or otherwise contacting an interior surface within the mouth. The head, and surface features of the head such as protrusions, ridges, dimples, brushes, bristles, holes, and/or indentations, may assist with removing surface material to expose the interior of the mouth to a gas, or moving soft tissue within the mouth to uncover or simply reach areas that are normally covered by tissue. For example, the outlets may deliver gas that includes oxygen between papillae of the tongue that are being physically moved by the action of the head on the tongue. The head (which may contain, carry, or transport chemicals and/or solutions) may be used to reach areas such as the roof of the mouth, the gums, the cheeks, surfaces below the tongue, and the inner surfaces of a person's lips.

The system may be used to deliver gas to a location being massaged, scraped, brushed, or otherwise being physically contacted as the contact occurs in a contact-plus-gas delivery procedure. The combination of gas delivery into the mouth at locations where scraping, brushing, massaging, or other physical contact is occurring while the contact occurs may permit interior surfaces of the mouth to be exposed to a greater concentration of oxygen than with other conventional systems. The greater incidence of exposure may improve the likelihood that bacteria associated with bad breath and/or halitosis will be killed by oxygen exposure, thus more effectively reducing or preventing bad breath and/or halitosis. Alternatively, the system may be used to simply deliver gas into a person's mouth during a flowing procedure that may last for one (1) to ten (10) minutes. In a more preferred embodiment, the duration would be three (3) to six (6) minutes. In a most preferred embodiment, the duration might be approximately five (5) minutes. Longer and shorter time periods may be used.

During this time, the user might simply allow their mouth to fill with gas that may flow through the mouth, gradually allowing more surfaces within the mouth to be exposed to oxygen for a greater period of time. The flowing or filling or saturating procedure may be used in conjunction with or separately from the contact-plus-gas delivery procedure.

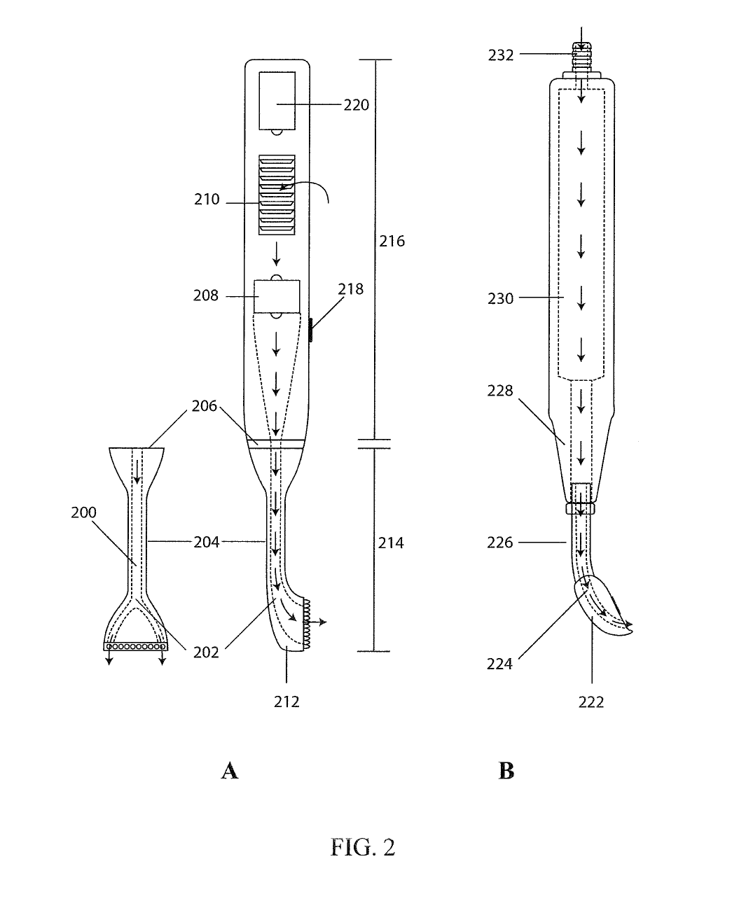

FIG. 2A is a cutaway view of the system, according to an embodiment. In the embodiment, the head (214) is removable, or interchangeable (204), from the body (216), and may be formed as a scraper head (200) or a brush head (212). The head attaches to the body at the attachment point (206). The scraper head may include a scraping end formed with an elongated scraping channel that extends along the Y-axis and includes a plurality of outlets. Each end of the scraping channel may be connected to a respective end of two branches of a forked gas delivery passage, or cannula (202). The forked gas delivery passage may converge to a single channel that extends along a mid-portion of the scraper head. The brush head may include a brushing end formed with a plurality of outlets formed between the bristles and/or protrusions of the brush head.

In the embodiment, the body is a handle that includes an on/off button (218), an air driving system (208), an air intake (210), and a battery (220), each of which are housed within the handle. The gas driving system may be a fan, as shown in FIG. 2A, or a gas compressor. The gas driving system is operable to pull air into the handle (or body) from the environment then through the handle (and/or body), into the head, and into a person's mouth.

FIG. 2B is an illustration of the system according to an embodiment. In the embodiment the scraping head (222) is detachable from the body (228). The scraping head may include a scraping end, with a plurality of outlets, formed with an elongated channel (226) that extends along the X-axis and includes an interior conduit (224). Each of the plurality of the outlets of the scraping head may be connected to a respective end of two branches of a forked gas delivery passage. The body contains a hollow interior chamber (230), which acts as a conduit allowing passage of at least one gas through it, and out through the scraping head. The hollow chamber acts as a conduit, with a connection port (232) for attaching a tube, pipe, or hose, to allow delivery of at least one gas.

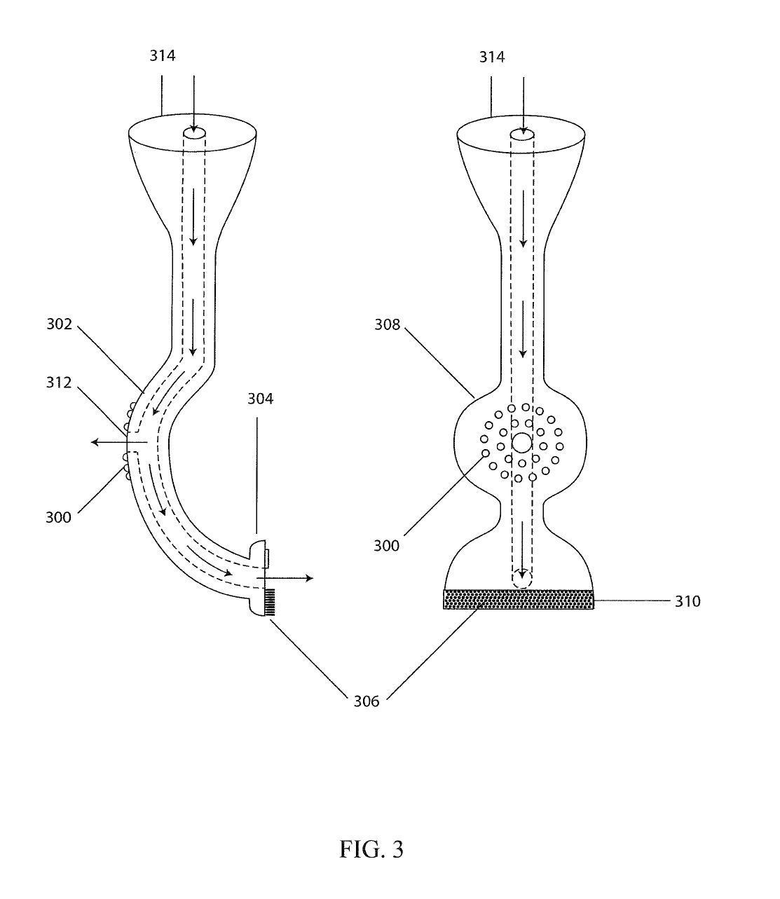

FIG. 3 is an illustration of an expanded width head of the system, according to an embodiment. The expanded width head includes a connecting end (314) for attaching the expanded width head to the body, an extension portion that extends along the X-axis from the connecting end, an arched portion that is connected to the extension portion (302) and is curved around an axis parallel to the Z-axis, and a distal end connected to the arched portion (304). As the arched portion is extended away from the connecting end, the arched portion is also extended along the Y-axis in a first direction before curving back to extend along the Y-axis in a second direction opposite the first direction. As the arched portion is extended away from the connecting end, the arched portion flares wider (308) along the Z-axis before narrowing.

The arched portion (302) may include protrusions (300), ridges, dimples, brushes, bristles, holes, and/or indentations on the surface facing the first direction along the Y-axis (e.g., the peaked portion which may be compared to the back of a spoon). The arched portion may include one or more outlets (312) for delivering gas to an interior surface of a person's mouth that is being physically contacted by the arched portion.

The distal end is distal to the connecting end, and connects to the arched portion at its narrowed location. As shown in FIG. 3, the distal end (304) includes a scraping and/or brushing end (310) with a single air passage that does not diverge before opening into multiple outlets. Alternatively, the distal end may include a scraping or a brushing head as discussed with respect to FIG. 2A. The distal end may include protrusions (306), ridges, dimples, brushes, bristles, holes, and/or indentations on a surface that faces the second direction along the Y-axis opposite the first direction. In some embodiments, the surface of the distal end and the surface of the arched portion may face directions that are angled with respect to each other rather than facing opposite directions. The connecting end, the extension portion, the arched portion, and the distal end cooperate to form a passage for transporting gas from the body to the outlets of the arched portion and the distal end.

The distal end of the expanded width head may be suitable for scraping a person's tongue and reaching into other areas, such as underneath the tongue, behind teeth, or the roof of a person's mouth. The arched portion may be suitable for rubbing against broad surfaces such as the interior walls of a person's cheeks.

Gas, such as oxygen or air containing oxygen, may be blown through the outlets of both the distal end and the arched portion simultaneously, thus performing both the functions of the flowing procedure and the contact-plus-gas delivery procedure. While one part of the interior of the mouth receives the benefit of the contact-plus-gas delivery procedure, the remaining portions of the mouth may receive the benefit of the gas emerging from the outlets not in proximity to an interior part of the person's mouth.

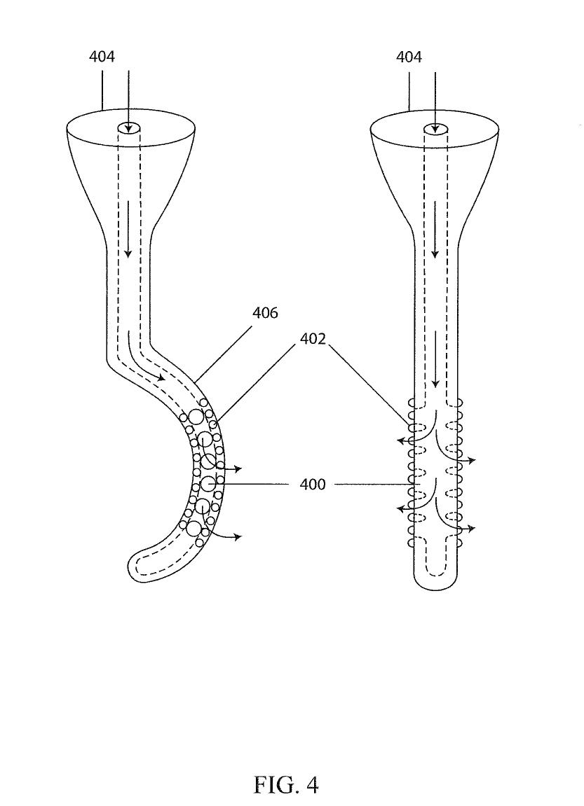

FIG. 4 is an illustration of a restricted width head of the system, according to an embodiment. The restricted width head may include a connecting end (404) for attaching the restricted width head to the body, an extension portion that extends along the X-axis from the connecting end, an arched portion (406) that is connected to the extension portion and curved around an axis parallel to the Z-axis, and a distal end connected to the arched portion. As the arched portion is extended away from the connecting end along the X-axis, the arched portion is also extended along the Y-axis in a first direction before curving back to extend along the Y-axis in a second direction opposite the first direction. As the arched portion is extended away from the connecting end, the arched portion in this embodiment is preferably substantially the same cross sectional shape (if taken in the Y-Z plane of FIG. 4).

Note that in FIG. 3, two views of the same device are given, the X-Y and the Y-Z views. The same is true for FIG. 4.

The arched portion may include surface features such as protrusions (402), ridges, dimples, brushes, bristles, holes (400), and/or indentations on the surface facing a third direction aligned with the Z-axis, and a fourth direction aligned with the Z-axis and oriented opposite to the third direction. In some embodiments, the surface feature may cover additional sides of the arched portion. The arched portion may include one or more outlets (400) for delivering gas to an interior surface of a person's mouth that is being physically contacted by the arched portion or through outlets that are not proximate to an inner surface of a person's mouth. The outlets may be on or between surface features of the arched portion.

The hooked and narrowed form of the arched portion may permit a user to reach into areas of the mouth such as between the gums and the lips, underneath and/or around the tongue. The use of surface features on two or more sides of the arched portion may permit the restricted width head to be used in different directions by flipping the restricted width head 180 degrees relative to a surface of the mouth.

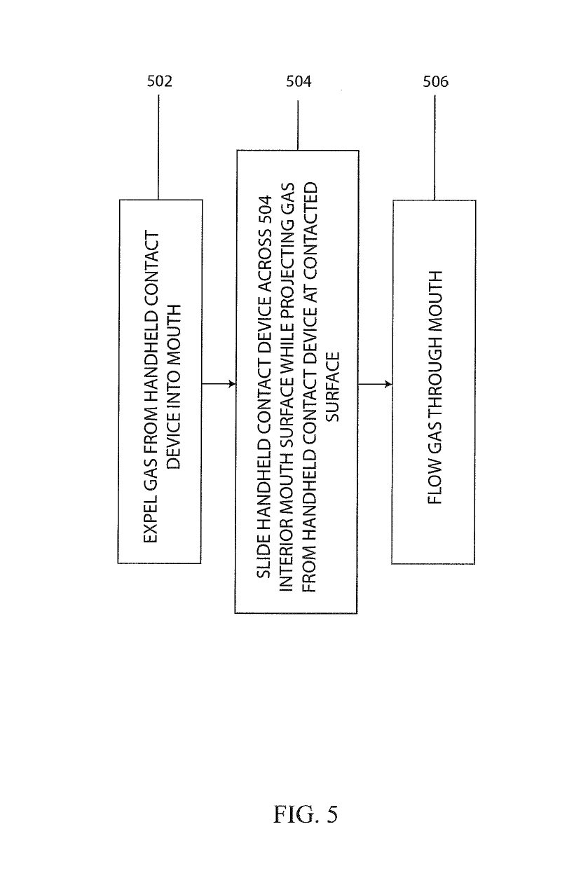

FIG. 5 is a flowchart illustrating operations of the method, according to an embodiment. The operations may be performed consecutively or with other operations between the illustrated operations. The operations may be performed in the order shown, or rearranged to be performed in alternate orders. The operations may be performed once, or repeated.

Prior to using the method, the person whose mouth to be treated may refrain from cleaning their mouth for a limited period of time, such as 30 minutes to 12 hours, so preferably no using toothpaste or over the counter mouthwashes. Toothpaste and mouthwash or the like may interfere with treatment of the person's mouth using the above described inventive systems and methods. The person may, however, rinse their mouth using antiseptic solutions used by dentists or oral surgeons prior to tooth cleaning or oral surgery.

In operation, a gas (502) is expelled from a handheld contact device into the mouth of a person. The handheld contact device may be the system shown in FIG. 1, FIG. 2A, FIG. 2B, FIG. 3, or FIG. 4. The gas preferably is oxygen, and it may be obtained from a tank of oxygen. In lesser-preferred embodiments, it may be air or air mixed with oxygen as discussed above, or other gas intended to reduce the number of bacteria in a person's mouth associated with bad breath and/or halitosis.

In operation, the handheld contact device (504) is controlled such that it slides across an interior mouth surface while projecting gas from the handheld contact device at the contacted surface. The handheld contact device may have a brush head, or a scraper head as shown in FIG. 2A and FIG. 2B, or it may be the expanded width head or restricted (narrow) width head of FIG. 3 and FIG. 4. The handheld contact device may be used on interior surfaces of the mouth such as the tongue, the teeth, the gums, the roof of the mouth, areas below and around the tongue, on the inside surfaces of a person's lips, and between the lips and a person's gums. The handheld contact device may include outlets that are not proximate to a surface of a user's mouth and that are used to project gas into the mouth.

Step (506) is for flowing a gas that includes oxygen into and through the person's mouth for a predetermined period of time to treat the interior surface of the person's mouth that has been contacted by the contact device. The gas may be the same as and provided from the source of FIG. 1 using the system of FIG. 1, FIG. 2A, FIG. 2B, FIG. 3, or FIG. 4. Most preferably, the gas is air with substantial oxygen or just pure oxygen. The handheld contact device may be held by the person's lips such that excess gas from the source is allowed to escape, but atmospheric gas does not enter the person's mouth. The flowing process may be performed simply by using a tube or hose that is also connected to the gas source.

The tube or hose may be held by a person's lips and/or teeth. The tube or hose may extend past the teeth to direct a flow of gas into the cavity mouth such that the opening of the tube or hose faces away from the person's teeth and/or lips that grip the hose or tube. The tube or hose may be directed into portions of a person's mouth, such as between the person's lips and gums, and/or the roof of the mouth. The tube or hose may be prevented from being directed at a person's teeth.

The velocity of the gas flowing from the hose or tube should be relatively slow (substantially slower) compared with the velocity of the air and/or oxygen that is emitted from the head. Therefore, it should be insufficient to dislodge any material from a person's teeth or from between a person's teeth because the purpose of providing the gas is to fill the person's mouth with a comfortable, continually refreshed source of oxygen that can saturate the interior cavity of a person's mouth with oxygen, thus permitting contact between bacteria for sufficient time and high enough concentrations of oxygen such that the bacteria will be killed, thus treating a potential source of bad breath and/or halitosis. The user may continue to breathe through his or her nose while oxygen or other gases are flowing through his or her mouth.

In some embodiments of the invention, such an input hose or tube may be used in conjunction with an output tube or hose. Instead of simply allowing gas to escape from a person's mouth, the outlet hose may be connected to a dental vacuum to allow the input gas to flow out of the person's mouth. Thus, the person's mouth may remain saturated with pure oxygen or other gases while the person breathes normally through their nose. Both the input hose and the output hose may be gripped by the person's teeth such that the outlets of each of the input hose and the output hose face away from a respective set of gripping teeth that grip either the input hose or the output hose (e.g., away from proximate teeth that grip the hose and towards the back of the mouth).

The person's mouth may be saturated with gas for seconds, minutes, or in less preferred embodiments, greater periods of time. This step of flowing the gas through the mouth or filling or saturating the mouth with gas is preferably continuous although in a lesser preferred embodiment it could be continual.

The extended exposure of the bacteria in the mouth to oxygen is believed to allow more effective treatment of bad breath and/or halitosis than use of liquids or mechanical scraping by themselves. Moreover, the use of positive pressure into the mouth is believed to allow gas containing oxygen to come into proximity with surfaces that may otherwise be inadequately exposed to treat the bacteria on or underneath those surfaces. The extended exposure of the mouth to a flow of gas is believed to further assist by drying the mouth as well as exposing additional bacteria to oxygen to further kill bacteria.

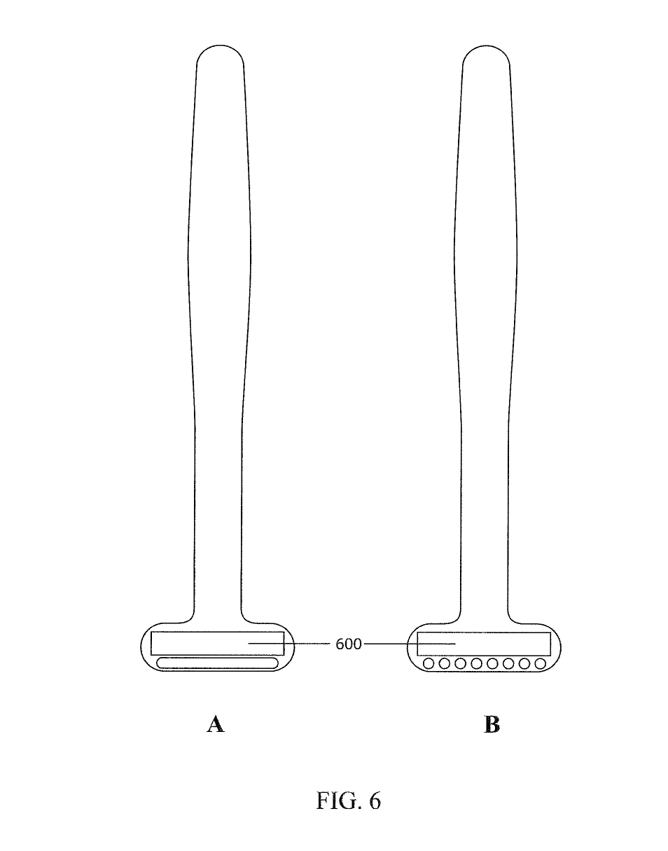

As shown in FIG. 6A and FIG. 6B, some embodiments of an oral hygiene system are shown to include a fluid dispensing material (600).

As shown in FIG. 6A, the system may be a standalone tongue scraper/wiper, cheek cleanser, and/or chemical delivery system that does not include mechanisms for delivering gas to the inside of a person's mouth. Such a system may be suitable for home use, travel, or wherever additional devices and systems may not be available.

The system of FIG. 6A may be used in combination with a separate gas supply, such as an oxygen tube that is held in the user's mouth. In such configurations, a vacuum tube may be used to remove excess gas and/or fluid from the person's mouth.

As shown in FIG. 6B, the system may include gas delivery components that can provide oxygen or other gasses to the inside of a person's mouth while the system is being used to wipe and/or scrape interior surfaces of a person's mouth (e.g., tongue, cheek, roof of mouth, and/or gums).

Structural Characteristics of Exemplary Systems: The system may include flexible surfaces and/or flexible components (not shown) to provide greater comfort to a user or more effective access to parts of a person's mouth. For example, the head of a tongue scraper may be rigid, such as being made from a hard plastic or metal. The tongue scraper may include a flexible scraper head, such as a flexible plastic bar supported at its ends, to allow better shaping of the material to a person's tongue or cheek.

In some embodiments, the head may be rigidly attached to the body, or it may be able to rotate and/or deflect relative to the body.

Surfaces of the system may be flat, concave, or convex, and may be smooth or have protrusions, indentations, and/or holes.

Fluid Dispensing Material Form: The fluid dispensing material may be formed as a strip, or the material may be shaped as separate pads that may be rounded, square, rectangular, or curved (not shown). The fluid dispensing material may be attached to or formed on protrusions on the head of the system, or the fluid dispersing material may be disposed in indentations, or disposed between indentations and/or protrusions on the head of the system (not shown).

The fluid dispensing material may be made of a similar material as the lubricant strips found on disposable razor heads. In other embodiments, the fluid dispensing material may be a sponge, hydrophilic rubber, fabric, or other material suitable for dispensing fluid. The fluid dispensing material may be adhered to, sleeved over, tied onto, clamped, screwed, pinched, or otherwise attached to the system.

The fluid may be retained in the fluid dispensing material such that when used on a person's tongue, the fluid is released. The fluid may be held by the fluid dispensing material for one time, or potentially multiple uses, prior to disposal of the fluid dispensing material and/or the system. The fluid dispensing material may hold dry chemicals that are mixed with a person's saliva before being released during use. In some embodiments, the user would dip the system and/or the strip into a chemical solution prior to using the system on the user's tongue.

Exemplary Fluids For Dispensing By The System: Exemplary fluids that can be dispensed by the system may be bactericidal, antibacterial, antiseptic, and/or antimicrobial. Bactericidal products destroy bacteria. Antibacterial products kill bacteria, or hinder their reproduction. Antiseptic products inhibit the growth and reproduction of many microorganisms, including bacteria, as well as fungi, protozoa, and viruses. Antimicrobial products are capable of destroying or inhibiting the growth of disease-causing microorganisms.

Although the invention has been described using specific terms, devices, and/or methods, such description is for illustrative purposes of the preferred embodiment(s) only. Changes may be made to the preferred embodiment(s) by those of ordinary skill in the art without departing from the scope of the present invention, which is set forth in the following claims. In addition, it should be understood that aspects of the preferred embodiment(s) generally may be interchanged in whole or in part.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

### REFERENCES

http://www.havlon.com/documents/Handbook_of_topical_microbials.pdf
http://onlinelibrary.wiley.com.ezproxy.csusm.edu/doi/10.1111/j.1601-0825.2005.01098.x/full#ss2

What is claimed is:

1. An oral device for a subject, comprising:
a. at least one body;
b. at least one scraping head; and
c. one or more of:
1) at least one gas; and
2) at least one reagent dispending material;
wherein said at least one body and at least one scraping head are operably engaged to apply physical contact of said at least one scraping head to the interior surfaces of a subject's oral cavity when in use.

2. The oral device of claim 1,
wherein said at least on body and said at least one scraping head are substantially hollow.

3. The oral device of claim 1,
wherein said at least one scraping head is detachable from said at least one body of said oral device.

4. The oral device of claim 1,
wherein said at least one scraping head comprises at least one aperture.

5. The oral device of claim 1,
wherein said oral device allows the flow of said at least one gas into said at least one body and exits from said at least one scraping head.

6. The oral device of claim 1,
wherein said at least one gas comprises oxygen.

7. The oral device of claim 1,
wherein said at least one reagent dispensing material faces generally outward from said oral device, said at least one body, said at least one scraping head, or a combination thereof.

8. The oral device of claim 1,
wherein said at least one reagent dispensing material comprises: at least one sponge, at least one hydrogel, at least one hydrogel with at least one dissolved gas, at least one hydrophilic rubber, at least one fabric, or a combination thereof.

9. The oral device of claim 1,
wherein the internal space of said oral device, said at least one body, said at least one scraping head, or a combination thereof, comprises at least one liquid phase.

10. The oral device of claim 9,
wherein said at least one liquid phase comprises: at least one antiseptic, at least one anti-microbial, at least one anti-fungal, at least one halogen-releasing antiseptic, at least one freshening agents, at least one bleach, at least one enzyme, at least one iodine, at least one numbing agent, or a combination thereof.

11. The oral device of claim 1,
wherein said oral device comprises at least one gas, at least one solution, at least one chemical, or a combination thereof.

12. The oral device of claim 11,
wherein said oral device drives the projection of said at least one gas, at least one solution, at least one chemical, or a combination thereof, away from said oral device towards the interior of the subject's oral cavity upon contact therewith.

13. A method of treating a subject for a disease, disorder, condition, or a combination thereof, of the oral cavity, comprising;
a) providing a subject in need of treatment of a disease, disorder, condition, or a combination thereof, of the oral cavity;
b) providing at least one oral device of claim 1;
c) engaging said subject with said at least one oral device;
wherein said subject is treated for said disease, disorder, condition, or a combination thereof, of the oral cavity.

14. The method of claim 13,
wherein said subject is a mammal, a primate, a non-human primate, or a human.

15. The method of claim 13,
wherein said disease, disorder, condition, or a combination thereof, comprises bad breath, halitosis, chronic halitosis, calculus, periodontitis, gum disease, fungal infections, dry mouth, or a combination thereof.

16. The method of claim 13,
wherein said disease, disorder, condition, or a combination thereof, comprises bad breath.

17. The method of claim 13,
wherein said subject's oral cavity is engaged with said at least one oral device.

18. The method of claim 13,
wherein said subject's oral cavity is contacted with said at least one oral device.

19. The method of claim 13,
wherein said at least one oral device administers at least one gas, at least one solution, at least one chemical, or a combination thereof to the oral cavity of said subject.

20. A method of treating a subject for a disease, disorder, condition, or a combination thereof, of the oral cavity, comprising;
a) providing a subject in need of treatment of a disease, disorder, condition, or a combination thereof, of the oral cavity;
b) providing at least one gas efficacious for the treatment of said disease, disorder, condition, or a combination thereof, of the oral cavity; and
c) providing at least one chemical efficacious for the treatment of said disease, disorder, condition, or a combination thereof, of the oral cavity;
wherein said subject is treated for said disease, disorder, condition, or a combination thereof, of the oral cavity.

21. The method of claim 20,
wherein said at least one gas comprises oxygen, nitrogen, nitrogen dioxide, hydrogen, helium, carbon dioxide, carbon monoxide, compressed air, nitrous oxide, or a combination thereof.

22. The method of claim 20,
wherein said at least one chemical comprises, at least one antiseptic, at least one antifungal, at least one antibiotic, at least one bleach, at least one iodine, at least one alcohol, at least one freshening agent, at least one hydrogen peroxide, at least one moisturizing agent, at least one salivating agent, at least one wetting agent, at least one numbing agent, or a combination thereof.

23. The method of claim 20,
wherein said method utilizes at least in part a device, at least in part a device of claim 1, or a combination thereof.
